(19) United States
(12) Patent Application Publication
(10) Pub. No.: US 2024/0254074 A1
(43) Pub. Date: Aug. 1, 2024

(54) METHOD FOR PURIFYING TRIALKYLAMINE, TRIALKYLAMINE PRODUCTION METHOD AND COMPOSITION

(71) Applicant: CENTRAL GLASS COMPANY, LIMITED, Yamaguchi (JP)

(72) Inventors: Ryosuke SAWAMURA, Yamaguchi (JP); Hiroyuki OOMORI, Yamaguchi (JP); Akiou KIKUCHI, Yamaguchi (JP); Naofumi TAMAI, Yamaguchi (JP); Takahisa TANIGUCHI, Yamaguchi (JP); Akifumi YAO, Yamaguchi (JP)

(73) Assignee: CENTRAL GLASS COMPANY, LIMITED, Yamaguchi (JP)

(21) Appl. No.: 18/019,682

(22) PCT Filed: Aug. 4, 2021
(86) PCT No.: PCT/JP2021/028882

§ 371 Date: Feb. 3, 2023

(30) Foreign Application Priority Data
Aug. 5, 2020 (JP) 2020-133282

(51) Int. Cl. C07C 209/84; C07C 211/04; C07C 211/05

(52) U.S. Cl. CPC C07C 209/84; C07C 211/04; C07C 211/05

(57) ABSTRACT

The present disclosure aims to provide a novel method for lowering the concentrations of dimethylamine, diethylamine, ethylpropylamine, and ethylisopropylamine in a crude trialkylamine. The present disclosure relates to a method for purifying a trialkylamine, including contacting a crude trialkylamine containing at least one impurity selected from the group consisting of dimethylamine, diethylamine, ethylpropylamine, and ethylisopropylamine with a zeolite to lower a concentration of the at least one impurity selected from the group consisting of dimethylamine, diethylamine, ethylpropylamine, and ethylisopropylamine in the crude trialkylamine than the concentration before contacting with the zeolite.

### TECHNICAL FIELD

The present disclosure relates to a method for purifying a trialkylamine using a zeolite, a method for producing a trialkylamine, and a composition containing a trialkylamine. The present disclosure particularly relates to a method for purifying triethylamine, a method for producing triethylamine, and a composition containing triethylamine, and a method for purifying trimethylamine, a method for producing trimethylamine, and a composition containing trimethylamine.

### BACKGROUND ART

A crude form of triethylamine, which is an organic amine, may contain diethylamine and other impurities. The purity of raw materials used in the production process of pharmaceutical products or the production process of semiconductor devices is preferably high. A reported method for purifying triethylamine is a method including: amidating impurities using an amidating agent such as acetyl chloride, into easily removable compounds; removing the amidated compounds by extraction with warm water; and refining triethylamine (see Patent Literature 1, for example).

The method disclosed in Patent Literature 1 requires multiple operations and processes including amidating and extracting impurities followed by refinement. There is a demand for a simpler method for purifying triethylamine without the use of additives such as amidating agents.

A crude form of trimethylamine, which is another organic amine, may contain dimethylamine in an amount of about 1000 ppm by volume as an impurity. Since trimethylamine and dimethylamine have close vapor pressures to be azeotropic, distillation can only lower the concentration of dimethylamine to about 400 to 500 ppm by volume.

Recently, a method using a synthetic zeolite has been proposed as a method for removing impurities in crude organic amines.

Patent Literature 2 discloses a purification apparatus for removing low-boiling impurities from monomethylamine by using a synthetic zeolite with pores of 0.3 nm or 0.4 nm in average diameter. Suggested examples of low-boiling impurities to be removed include hydrogen, oxygen, nitrogen, carbon monoxide, carbon dioxide, and methane. Patent Literature 2 further discloses adsorption of water and hydrocarbons using a synthetic zeolite with pores of 0.5 nm in average diameter.

Patent Literature 3 discloses a method for purifying trimethylamine in which a zeolite 3A or 4A is used to remove water and ammonia from a trimethylamine-containing gas and a zeolite 5A is used to remove monomethylamine and nitrogen from the trimethylamine-containing gas.

### CITATION LIST

### Patent Literature

Patent Literature 1: JP S53-01504 B (JP 934266 B)
Patent Literature 2: JP 2016-150926 A (JP 6441116 B)
Patent Literature 3: U.S. Pat. No. 8,664,446

### SUMMARY OF INVENTION

### Technical Problem

Patent Literature 3 only discloses a method for removing monomethylamine using a synthetic zeolite, and does not disclose the removal of diethylamine, ethylpropylamine, ethylisopropylamine, and dimethylamine using a synthetic zeolite. Therefore, the removal of diethylamine, ethylpropylamine, ethylisopropylamine, and dimethylamine using a synthetic zeolite has not known.

In view of the above issue, the present disclosure aims to provide a novel method for lowering the concentrations of dimethylamine, diethylamine, ethylpropylamine, and ethylisopropylamine in a crude trialkylamine.

### Solution to Problem

The present inventors made intensive studies to find that the concentrations of dimethylamine, diethylamine, ethylpropylamine, and ethylisopropylamine in an impurity-containing crude trialkylamine can be lowered by contacting the crude trialkylamine with a zeolite. Thus, the present disclosure was completed.

Specifically, a first method for purifying a trialkylamine of the present disclosure includes: contacting a crude trialkylamine containing at least one impurity selected from the group consisting of diethylamine, ethylpropylamine, and ethylisopropylamine with a zeolite to lower a concentration of the at least one impurity selected from the group consisting of diethylamine, ethylpropylamine, and ethylisopropylamine in the crude trialkylamine than the concentration before contacting with the zeolite.

The first method for purifying a trialkylamine of the present disclosure does not require a complicated process using additives and enables the removal of diethylamine, ethylpropylamine, and ethylisopropylamine to increase the purity of the trialkylamine by a simple operation.

A second method for purifying a trialkylamine of the present disclosure includes: contacting a crude trialkylamine containing at least dimethylamine as an impurity with a zeolite having pores of 0.2 to 0.6 nm in diameter to lower a concentration of the dimethylamine in the crude trialkylamine than the concentration before contacting with the zeolite.

The second method for purifying a trialkylamine of the present disclosure does not require a complicated process using additives and enables the removal of dimethylamine to increase the purity of the trialkylamine by a simple operation.

A method for producing a trialkylamine of the present disclosure includes: synthesizing a crude trialkylamine; and purifying the crude trialkylamine by the above purification method.

The method for producing a trialkylamine of the present disclosure enables the production of a trialkylamine with high purity by a simple operation.

A first composition of the present disclosure contains: a trialkylamine in an amount of 99.9 GC area % or more; and diethylamine in an amount of 1 GC area ppm or more and 150 GC area ppm or less.

A second composition of the present disclosure contains: a trialkylamine in an amount of 99.9 GC area % or more; and ethylpropylamine in an amount of 1 GC area ppm or more and 150 GC area ppm or less.

A third composition of the present disclosure contains: a trialkylamine in an amount of 99.9 GC area % or more; and ethylisopropylamine in an amount of 1 GC area ppm 5 or more and 20 GC area ppm or less.

The units “GC area %” and “GC area ppm” indicate the proportion of the area of the peaks derived from the component to be quantified in the total area of the peaks obtained by gas chromatography (GC). 10

A fourth composition of the present disclosure contains: a trialkylamine in an amount of 99.9% by weight or more; and dimethylamine in an amount of 50 ppm by volume or more and 400 ppm by volume or less.

A fifth composition of the present disclosure contains: a trialkylamine in an amount of 99.9% by weight or more; and dimethylamine in an amount of 10 ppm by volume or less.

The first to fifth compositions of the present disclosure can be used in the production process of pharmaceutical products and the production process of semiconductors which require high-purity raw materials.

### Advantageous Effects of Invention

The method for purifying a trialkylamine of the present disclosure enables the removal of impurities in a crude trialkylamine to increase the concentration of the trialkylamine by a simple operation without the use of additives, thereby supplying an extremely pure trialkylamine that can be used in the production process of pharmaceutical products and the production process of semiconductors.

### DESCRIPTION OF EMBODIMENTS

Hereinafter, the present disclosure is described in detail. The following descriptions of constituent elements are examples of embodiments of the present disclosure. The present disclosure is not limited to these specific descriptions. Various modifications can be made within the scope of the gist of the present disclosure.

### <First Method for Purifying Trialkylamine>

The first method for purifying a trialkylamine of the present disclosure includes contacting a crude trialkylamine containing at least one impurity selected from the group consisting of diethylamine, ethylpropylamine, and ethylisopropylamine with a zeolite to lower the concentration of the at least one impurity selected from the group consisting of diethylamine, ethylpropylamine, and ethylisopropylamine in the crude trialkylamine than the concentration before contacting with the zeolite.

Zeolites used in the method of the present disclosure are crystalline aluminosilicates, also called molecular sieves, and are classified as synthetic zeolites, artificial zeolites, and natural zeolites. In the method of the present disclosure, any of synthetic zeolites, artificial zeolites, or natural zeolites can be used. Preferably, a synthetic zeolite with high purity is used.

In the present disclosure, the zeolite used is preferably a zeolite having pores of 0.2 to 1.2 nm in diameter or 3 to 12 Å in diameter. When the diameter of the zeolite pores is outside the above range, the effect of reducing impurities in a crude triethylamine may be lowered.

The synthetic zeolite having pores of 0.2 to 1.2 nm in diameter is suitably a synthetic zeolite 3A, 4A, 5A, or 13X. The character “A” of synthetic zeolite 3A, 4A, or 5A stands for Å (angstrom). The pores of a synthetic zeolite 13X are 1.0 nm or 10 Å in diameter.

A synthetic zeolite 3A has a pore size of 0.3 nm and can pass molecules up to 0.3 nm in diameter. A synthetic zeolite 4A has a pore size of 0.35 nm but it can pass molecules up to 0.4 nm in diameter owing to the expansion/contraction and kinetic energy of the molecules coming into the cavities at normal operating temperatures. Similarly, a synthetic zeolite 5A has a pore size of 0.42 nm but it can pass molecules up to 0.5 nm in diameter for the same reason. A synthetic zeolite 13X has a pore size of 1.0 nm and can pass molecules up to 1.0 nm in diameter. The range of diameters (adsorption diameters) of molecules that can pass through synthetic zeolite 3A, 4A, 5A, or 13X is specified below.
Type 3A: 0.3 nm or less
Type 4A: 0.4 nm or less
Type 5A: 0.5 nm or less
Type 13X: 1.0 nm or less

Specific examples of usable synthetic zeolites 3A, 4A, 5A, and 13X include Molecular Sieve 3A, Molecular Sieve 4A, Molecular Sieve 5A, and Molecular Sieve 13X all available from Union Showa K. K. and Molecular Sieves 3A, Molecular Sieves 4A, Molecular Sieves 5A, and Molecular Sieves 13X all available from FUJIFILM Wako Pure Chemicals Corporation.

In the present disclosure, the zeolite used is more preferably a zeolite having pores of 0.8 to 1.2 nm in diameter, still more preferably a zeolite having pores of 0.9 to 1.1 nm in diameter.

A zeolite having pores of 0.8 to 1.2 nm in diameter is highly effective in reducing diethylamine, ethylpropylamine, and ethylisopropylamine in a crude trialkylamine. In the present disclosure, the zeolite used is particularly preferably a synthetic zeolite 13X.

The zeolite used in the method of the present disclosure may have any form such as beads, pellets, or powder. A zeolite in the form of beads or pellets is preferred because it is readily usable in chemical plants.

The zeolite used in the method of the present disclosure may be purchased and used as is. Preferably, the zeolite is dried prior to contacting with a crude trialkylamine. The drying condition is preferably one hour or longer at 100° C. or higher, more preferably one to two hours at 100° ° C. to 150° C.

The method for contacting a crude trialkylamine with a zeolite is not limited. Examples thereof include a method including adding a zeolite to a container containing a crude trialkylamine and leaving them to stand (immersion method) and a method including packing a column or a packing tower with a zeolite and allowing a crude trialkylamine to pass through the column or packing tower to contact with the zeolite (column method). In the method of the present disclosure, the immersion method is preferred because of its simplicity.

Examples of the trialkylamine include amines in which three alkyl groups are C1-C4 alkyl groups. Examples of C1-C4 alkyl groups in a trialkylamine include methyl, ethyl, n-propyl, isopropyl, butyl, isobutyl, and t-butyl groups. The three alkyl groups in the trialkylamine may be groups which are the same as or different from one another. Preferably, they all are the same groups.

Examples of the trialkylamine include trimethylamine, triethylamine, tri-n-propylamine, triisopropylamine, tributylamine, triisobutylamine, and tri-t-butylamine.

A crude trialkylamine may be contacted with a zeolite under any conditions, such as at 0° ° C. to 80ºC for 30 seconds to 24 hours. When the crude trialkylamine is crude triethylamine, the contacting temperature is preferably 10° C. or higher and 40° C. or lower. Crude triethylamine is liquid at room temperature at normal pressure. More preferably, crude triethylamine in a liquid state is contacted with a zeolite at 10° C. or higher and 40° ° C. or lower.

The first method for purifying a trialkylamine of the present disclosure is suitable for the purification of triethylamine. Crude triethylamine, one of the targets to be purified by the method of the present disclosure, is triethylamine containing at least one of diethylamine, ethylpropylamine, or ethylisopropylamine as an impurity. Crude triethylamine may be purchased or obtained by synthesizing triethylamine by a conventionally known method. How it is obtained is not limited. Examples of methods for synthesizing triethylamine include the acetaldehyde method including reacting acetaldehyde, ammonia, and hydrogen using a catalyst, and the ethyl alcohol method in which the acetaldehyde in the acetaldehyde method is replaced by ethyl alcohol.

The crude trialkylamine may contain other impurities besides diethylamine, ethylpropylamine, and ethylisopropylamine. Examples of the other impurities include monoethylamine, acetaldehyde, ethyl alcohol, hydrogen, oxygen, nitrogen, carbon monoxide, carbon dioxide, methane, ammonia, and water.

The crude trialkylamine preferably contains a trialkylamine in an amount of 99 GC area % or more, more preferably in an amount of 99.5 GC area % or more.

The crude trialkylamine contains at least diethylamine as an impurity. The amount of diethylamine is preferably more than 150 GC area ppm, more preferably more than 150 GC area ppm and 250 GC area ppm or less.

The crude trialkylamine preferably contains ethylpropylamine in an amount of more than 150 GC area ppm or contains ethylisopropylamine in an amount of more than GC area ppm. More preferably, the crude trialkylamine contains ethylpropylamine in an amount of more than 150 GC area ppm and 250 GC area ppm or less and ethylisopropylamine in an amount of more than 20 GC area ppm and 30 GC area ppm or less.

If the concentration of diethylamine, ethylpropylamine, or ethylisopropylamine in the crude trialkylamine is greater than the above corresponding value, these impurities may be removed by distillation or other methods prior to the treatment by the method of the present disclosure.

In the method of the present disclosure, the crude trialkylamine is contacted with a zeolite to lower the concentration of at least one of diethylamine, ethylpropylamine, and ethylisopropylamine in the crude trialkylamine than the concentration before contacting with the zeolite.

In the method of the present disclosure, the preferred concentration of each component after contacting with the zeolite is specified below.

The purity of the trialkylamine is preferably 99.9 GC area % or higher, more preferably 99.95 GC area % or higher.

Diethylamine in the trialkylamine is preferably 150 GC area ppm or less, more preferably 100 GC area ppm or less.

Ethylpropylamine in the trialkylamine is preferably 150 GC area ppm or less, more preferably 100 GC area ppm or less.

Ethylisopropylamine in the trialkylamine is preferably 20 GC area ppm or less, more preferably 15 GC area ppm or less.

The purification method of the present disclosure can also lower the concentrations of impurities other than diethylamine, ethylpropylamine, and ethylisopropylamine.

### <Second Method for Purifying Trialkylamine>

The details of the second method for purifying a trialkylamine will be described in the following. Only the points different from the first method for purifying a trialkylamine are described, and descriptions on common contents are omitted.

The second method for purifying a trialkylamine of the present disclosure includes contacting a crude trialkylamine containing at least dimethylamine as an impurity with a zeolite having pores of 0.2 to 0.6 nm in diameter to lower the concentration of the dimethylamine in the crude trialkylamine than the concentration before contacting with the zeolite.

In the second method for purifying trimethylamine, the zeolite used needs to be a zeolite having pores of 0.2 to 0.6 nm in diameter. When the diameter of the zeolite pores is less than 0.2 nm or greater than 0.6 nm, the effect of lowering the concentration of dimethylamine in the crude trialkylamine is reduced. A synthetic zeolite 3A, 4A, or 5A is suitable as a synthetic zeolite having pores of 0.2 to 0.6 nm in diameter.

The zeolite used in the method of the present disclosure may be purchased and used as is. Preferably, the zeolite is dried prior to contacting with a crude trialkylamine. The drying condition is preferably 30 minutes or longer at 1 kPa or less at 150° C. or higher, more preferably 30 to 60 minutes at 1 kPa or less at 150° ° C. to 200° C.

The method for contacting a crude trialkylamine with a zeolite is not limited. Preferred is the column method in terms of its high effect of removing dimethylamine as an impurity and capability of purification in a short time. However, when the concentration of dimethylamine in the crude trialkylamine is high, the immersion method and the column method can be combined. For example, the immersion method is carried out before the column method to lower the concentration of dimethylamine to a certain level.

Especially when the crude trialkylamine is crude trimethylamine, the temperature and pressure conditions for flow contact of crude trimethylamine with a zeolite are preferably 20° C. to 30° C. at atmospheric pressure or higher. The pressure condition is more preferably 150 to 200 kPa. The time for flow contact of crude trimethylamine with a zeolite is preferably 50 to 200 seconds. When the time for contact is less than 50 seconds, dimethylamine may not be sufficiently removed. In contrast, flow contact for more than 200 seconds may not any more increase the effect of removing dimethylamine. The time for flow contact of crude trimethylamine with a zeolite is more preferably 100 to 150 seconds. The second method for purifying a trialkylamine of the present disclosure is suitable for the purification of trimethylamine.

In a preferred embodiment of the second method for purifying a trialkylamine, crude trimethylamine in a gaseous state is flowed to contact with a zeolite at 20° C. to 30° ° C. at atmospheric pressure or higher for 100 seconds or longer.

When crude trimethylamine is flowed to contact with a zeolite, the linear velocity of the crude trimethylamine is preferably 0.001 to 0.1 m/sec, more preferably 0.01 to 0.1 m/sec.

Crude trimethylamine, which is one of the targets to be purified by the method of the present disclosure, is trimethylamine containing at least dimethylamine as an impurity. Crude trimethylamine may be purchased or obtained by synthesizing trimethylamine by a conventionally known method (e.g., the method described in JP S58-049340 A). How it is obtained is not limited.

In the second purification method, the crude trialkylamine preferably contains a trialkylamine in an amount of 98% by weight, more preferably 99% by weight or more, still more preferably 99.9% by weight or more.

The concentration of dimethylamine in the crude trialkylamine is preferably 500 to 1500 ppm by volume. When the concentration of dimethylamine in the crude trialkylamine is greater than the above value, dimethylamine may be removed by distillation or other methods prior to the treatment by the method of the present disclosure, or the static method and the column method may be combined.

Dimethylamine in the crude trialkylamine is unstable and its concentration in the gaseous phase is not stable.

In a preferred embodiment, the crude trialkylamine to be purified by the method of the present disclosure may be a liquid or a gas. When the crude trialkylamine is crude trimethylamine, gaseous crude trimethylamine is preferred because purification can be performed at room temperature and normal pressure.

In the second purification method of the present disclosure, the concentration of dimethylamine in the crude trialkylamine is preferably lowered to 400 ppm by volume or less, more preferably to 300 ppm by volume or less. The lower limit of the concentration of dimethylamine in the trialkylamine purified by the method of the present disclosure is, for example, 50 ppm by volume or 90 ppm by volume. Combination of the second purification method of the present disclosure with various purification methods, such as known distillation methods including single distillation, continuous distillation, or precise distillation can greatly lower the concentration of dimethylamine in the trialkylamine to 10 ppm by volume or less. The concentration of dimethylamine can be lowered to below the detection limit. In such a case, the lower limit of the concentration of dimethylamine in the trialkylamine is, for example, an amount greater than 0 ppm by volume. Use of the method of the present disclosure can provide a trialkylamine with high purity as described above. Such a trialkylamine with high purity is suitable for applications such as dry etching of silicon oxides.

The present disclosure also relates to a method for producing a trialkylamine, including: synthesizing a crude trialkylamine; and purifying the crude trialkylamine by the first method for purifying a trialkylamine and/or the second method for purifying a trialkylamine. In the step of purifying the crude trialkylamine, both the first method for purifying a trialkylamine and the second method for purifying a trialkylamine may be used.

The step of synthesizing a crude trialkylamine can be performed, for example, by the method described above for the first method for purifying a trialkylamine and the second method for purifying a trialkylamine, but the method is not limited thereto.

The step of purifying the crude trialkylamine can be performed in the same manner using the materials, operations, and procedures described above for the first method for purifying a trialkylamine and the second method for purifying a trialkylamine.

In the trialkylamine obtained by the production method of the present disclosure, the concentration of at least one of dimethylamine, diethylamine, ethylpropylamine, and ethylisopropylamine should be lower than the concentration thereof in the crude trialkylamine before contacting with the zeolite. The concentration of each component is as described above for the methods.

The first composition of the present disclosure contains a trialkylamine in an amount of 99.9 GC area % or more and diethylamine in an amount of 1 GC area ppm or more and 150 GC area ppm or less. The amount of diethylamine in the first composition is preferably 1 GC area ppm or more and 100 GC area ppm or less.

The second composition of the present disclosure contains a trialkylamine in an amount of 99.9 GC area % or more and ethylpropylamine in an amount of 1 GC area ppm or more and 150 GC area ppm or less. The amount of ethylpropylamine in the second composition is preferably 1 GC area ppm or more and 100 GC area ppm or less.

The third composition of the present disclosure contains a trialkylamine in an amount of 99.9 GC area % or more and ethylisopropylamine in an amount of 1 GC area ppm or more and 20 GC area ppm or less. The amount of ethylisopropylamine in the third composition is preferably 1 GC area ppm or more and 15 GC area ppm or less.

The fourth composition of the present disclosure contains a trialkylamine in an amount of 99.9% by weight or more and dimethylamine in an amount of 50 ppm by volume or more and 400 ppm by volume or less. The amount of dimethylamine in the fourth composition is preferably 90 ppm by volume or more and 300 ppm by volume or less.

The fifth composition of the present disclosure contains a trialkylamine in an amount of 99.9% by weight or more and dimethylamine in an amount of 10 ppm by volume or less. The amount of dimethylamine in the fifth composition is preferably more than 0 ppm by volume and 10 ppm by volume or less.

These first to fifth compositions can be obtained by the method for producing a trialkylamine described above.

### EXAMPLES

The following shows examples more specifically describing embodiments of the present disclosure. The present disclosure is not limited to these examples only. The Molecular Sieves 3A, 4A, 5A, and 13X used in the examples correspond to synthetic zeolites 3A, 4A, 5A, and 13X, respectively.

### (Purification of Crude Triethylamine)

Crude triethylamine used in the present examples was synthesized with reference to a conventional production method. The purity of triethylamine in the crude triethylamine obtained by the synthesis was 99.89 GC area %. The crude triethylamine contained impurities including diethylamine in an amount of 155.9 GC area ppm, ethylpropylamine in an amount of 179.3 GC area ppm, and ethylisopropylamine in an amount of 20.8 GC area ppm.

The concentrations of triethylamine and impurities were analyzed using a gas chromatograph analyzer under the following conditions. With the retention time of triethylamine taken as 1.00, the total area of the gas chromatography chart of components having a relative retention time of 1.85 or less was determined. Based on the obtained total area set to 100 GC area %, the GC area % concentration or GC area ppm concentration of each component was determined. The relative retention times of diethylamine, ethylpropylamine, and ethylisopropylamine are 0.69, 0.95, and 0.84, respectively.

### (Gas Chromatography Analysis Conditions)

Sample: 1 μL of liquid crude triethylamine
Instrument: GC-2014 (Shimadzu Corporation)
Detector: flame ionization detector (FID)
Column used: Inertcap for Amines (60 m in length x 0.32 mm in internal diameter, available from G.L. Science Inc.)
Analysis condition: Injection temperature 200° C., detection temperature 250° C.
Carrier gas: Helium
Column flow rate: 2.99 mL/min
Column temperature: retention at 50° ° C. for 15 minutes, temperature rise to 200° C. at 10° C./min, retention at 200° ° C. for 5 minutes, temperature rise to 250° C. at 10° C./min, and retention at 250° ° C. for 5 minutes

### Example 1

A glass container was charged with 10 mL of crude triethylamine, and 5 g of Molecular Sieve 3A (pore size: 0.3 nm, available from Union Showa K. K.) dried by heating in a drying oven at 120ºC for one hour was added thereto to be immersed in the crude triethylamine, followed by standing for one minute. Then, the concentrations of triethylamine and impurities obtained from the immersion treatment were analyzed using a gas chromatograph analyzer. The results are shown in Table 1.

### Example 2

The immersion treatment was carried out as in Example 1 except that Molecular Sieve 3A was replaced with Molecular Sieve 4A (pore size: 0.35 nm, available from Union Showa K. K.), and the concentrations of triethylamine and impurities were analyzed. The results are shown in Table 1.

### Example 3

The immersion treatment was carried out as in Example 1 except that Molecular sieve 3A was replaced with Molecular Sieve 5A (pore size: 0.42 nm, available from Union Showa K.K.), and the concentrations of triethylamine and impurities were analyzed. The results are shown in Table 1.

### Example 4

The immersion treatment was carried out as in Example 1 except that Molecular Sieve 3A was replaced with Molecular Sieve 13X (pore size: 1.0 nm, available from FUJIFILM Wako Pure Chemicals Corporation), and the concentrations of triethylamine and impurities were analyzed. The results are shown in Table 1.

### Comparative Example 1

Without the immersion treatment, the concentrations of crude triethylamine and impurities were analyzed using a gas chromatograph analyzer. The results are shown in Table 1.

TABLE 1

| | | Triethylemine | Diethylamine | Ethylpropylamine | Ethylisopropylamine |
|---|---|---|---|---|---|
| | Zeolite | (GC area %) | (GC area ppm) | (GC area ppm) | (GC area ppm) |
| Comparative | Not used | 99.89 | 155.9 | 179.3 | 20.8 |
| Example 1 | | | | | |
| Example 1 | 3A | 99.91 | 130.4 | 122.5 | 19.0 |
| Example 2 | 4A | 99.92 | 124.1 | 118.3 | 18.5 |
| Example 3 | 5A | 99.91 | 134.5 | 125.2 | 19.1 |
| Example 4 | 13X | 99.96 | 63.9 | 77.1 | 14.2 |

As shown in Table 1, the concentration of triethylamine after the immersion treatment was higher and the concentrations of diethylamine, ethylpropylamine, and ethylisopropylamine were lower in Examples 1 to 4 than in Comparative Example 1 in which the immersion treatment was not carried out. In Example 4 in which Molecular Sieve 13X was used as the zeolite, the concentrations of diethylamine and ethylpropylamine were less than half the concentrations in Comparative Example 1 and the concentration of ethylisopropylamine was lower than the concentration in Comparative Example 1 by 30% or more.

### (Purification of Crude Trimethylamine)

Crude trimethylamine used in the examples was synthesized with reference to a conventional production method. The trimethylamine contained 500 to 2000 ppm by volume of dimethylamine and 100 to 1000 ppm by volume of water as impurities in the gaseous phase. Impurity concentrations were analyzed using a gas chromatograph analyzer (GC-2014, available from Shimadzu Corporation, Detector: FID).

### Example 5

One packing tower of 10.6 mm in diameter and 0.1 m in length was packed with Molecular Sieve 3A (pore size: 0.3 nm, available from Union Showa K. K.) as a zeolite, followed by drying under reduced pressure at 150ºC for 30 minutes. Then, crude trimethylamine was passed through the tower at a linear velocity of 0.02 m/sec (contact time with zeolite: seconds). Trimethylamine was collected from the outlet of the packing tower, and the concentration of dimethylamine was analyzed. The results are shown in Table 2.

### Example 6

Crude trimethylamine was passed through the tower as in Example 5 except that the length of the packing tower was changed to 1 m (contact time with zeolite: 50 seconds), and the concentration of dimethylamine was analyzed. The results are shown in Table 2.

### Example 7

Crude trimethylamine was passed through the tower as in Example 6 except that the number of packing towers was changed to two (contact time with zeolite: 100 seconds), and the concentration of dimethylamine was analyzed. The results are shown in Table 2.

### Example 8

Crude trimethylamine was passed through the tower as in Example 6 except that the number of packing towers was changed to three (contact time with zeolite: 150 seconds), and the concentration of dimethylamine was analyzed. The results are shown in Table 2.

### Example 9

Crude trimethylamine was passed through the tower as in Example 5 (contact time with zeolite: 5 seconds) except that the zeolite was changed to Molecular Sieve 4A (pore size: 0.35 nm, available from Union Showa K. K.), and the concentration of dimethylamine was analyzed. The results are shown in Table 2.

### Example 10

Crude trimethylamine was passed through the tower as in Example 9 except that the length of the packing tower was changed to 1 m (contact time with zeolite: 50 seconds), and the concentration of dimethylamine was analyzed. The results are shown in Table 2.

### Example 11

Crude trimethylamine was passed through the tower as in Example 10 except that the number of packing towers was changed to two (contact time with zeolite: 100 seconds), and the concentration of dimethylamine was analyzed. The results are shown in Table 2.

### Example 12

Crude trimethylamine was passed through the tower as in Example 10 except that the number of packing towers was changed to three (contact time with zeolite: 150 seconds), and the concentration of dimethylamine was analyzed. The results are shown in Table 2.

### Example 13

Crude trimethylamine was passed through the tower as in Example 5 (contact time with zeolite: 5 seconds) except that the zeolite was changed to Molecular Sieve 5A (pore size: 0.42 nm, available from Union Showa K. K.), and the concentration of dimethylamine was analyzed. The results are shown in Table 2.

### Example 14

Crude trimethylamine was passed through the tower as in Example 13 except that the length of the packing tower was changed to 1 m (contact time with zeolite: 50 seconds), and the concentration of dimethylamine was analyzed. The results are shown in Table 2.

### Example 15

Crude trimethylamine was passed through the tower as in Example 14 except that the number of packing towers was changed to two (contact time with zeolite: 100 seconds), and the concentration of dimethylamine was analyzed. The results are shown in Table 2.

### Example 16

Crude trimethylamine was passed through the tower as in Example 14 except that the number of packing towers was changed to three (contact time with zeolite: 150 seconds), and the concentration of dimethylamine was analyzed. The results are shown in Table 2.

### Comparative Example 2

Crude trimethylamine was passed through the tower as in Example 5 except that the packing tower was not packed with zeolite, and the concentration of dimethylamine was analyzed. The results are shown in Table 2.

### Comparative Example 3

Crude trimethylamine was passed through the tower as in Example 5 (contact time with zeolite: 5 seconds) except that the zeolite was changed to Molecular Sieve 13X (pore size: 1.0 nm, available from Union Showa K.K.), and the concentration of dimethylamine was analyzed. The results are shown in Table 2.

### Comparative Example 4

Crude trimethylamine was subjected to total reflux through a column with 20 theoretical plates for 72 hours, and trimethylamine in an amount of 30% by weight of the charged amount was purged from the top of the column at a reflux ratio of 200. Then, the concentration of dimethylamine in trimethylamine as a liquid phase was analyzed. The results are shown in Table 2.

TABLE 2

| | | | Concentration of |
|---|---|---|---|
| | | Contact time | dimethylamine |
| | Filler | (sec) | (ppm by volume) |
| Example 5 | Molecular Sieve | 5 | 740 |
| Example 6 | 3A | 50 | 270 |
| Example 7 | | 100 | 120 |
| Example 8 | | 150 | 140 |
| Example 9 | Molecular Sieve | 5 | 310 |
| Example 10 | 4A | 50 | 230 |
| Example 11 | | 100 | 190 |
| Example 12 | | 150 | 160 |
| Example 13 | Molecular Sieve | 5 | 380 |
| Example 14 | 5A | 50 | 140 |
| Example 15 | | 100 | 110 |
| Example 16 | | 150 | 100 |
| Comparative | None | — | 1200 |
| Example 2 | | | |
| Comparative | Molecular Sieve 13X | 5 | 1100 |
| Example 3 | | | |
| Comparative | None (distillation) | — | 427 |
| Example 4 | | | |

As shown in Table 2, the concentration of dimethylamine after purification in Examples 5 to 16 was significantly lower than the concentration in Comparative Example 2 in which no filler was used. The concentration of dimethylamine was lower in Examples 6 to 8, 10 to 12, and 14 to 16 in which the contact time with zeolite was 50 seconds or longer than in Comparative Example 4 in which distillation was performed. The concentration of dimethylamine in Examples 7, 8, 11, 12, 15, and 16 in which the contact time with the zeolite was 100 seconds or longer was half or less of the concentration in Comparative Example 4. In Comparative Example 3 in which Molecular Sieve 13X was used as the zeolite, the concentration of dimethylamine was almost the same as the concentration in Comparative Example 2 in which no filler was used.

1-12. (canceled)

13. A method for purifying a trialkylamine, comprising: contacting a crude trialkylamine containing at least dimethylamine as an impurity with a zeolite having pores of 0.2 to 0.6 nm in diameter to lower a concentration of the dimethylamine in the crude trialkylamine than the concentration before contacting with the zeolite.

14. The method for purifying a trialkylamine according to claim 13, wherein the concentration of the dimethylamine in the crude trialkylamine is lowered to 400 ppm by volume or less.

15. The method for purifying a trialkylamine according to claim 13, wherein the zeolite is dried at 1 kPa or less at 150° C. or higher for 30 minutes or longer prior to contact with the crude trialkylamine.

16. The method for purifying a trialkylamine according to claim 13, wherein all three alkyl groups in the trialkylamine are the same groups.

17. The method for purifying a trialkylamine according to claim 13, wherein the trialkylamine is trimethylamine.

18. The method for purifying a trialkylamine according to claim 17, wherein a crude trimethylamine in a gaseous state is flowed to contact with the zeolite at 20° ° C. to 30° C. and at atmospheric pressure or higher for 100 seconds or longer.

19. The method for purifying a trialkylamine according to claim 13, wherein the crude trialkylamine before contacting with the zeolite has a concentration of the dimethylamine of 500 to 1500 ppm by volume.

20. A method for producing a trialkylamine, comprising: synthesizing a crude trialkylamine; and purifying the crude trialkylamine by the method according to claim 13.

21-25. (canceled)

26. A composition comprising: a trialkylamine in an amount of 99.9% by weight or more; and dimethylamine in an amount of 50 ppm by volume or more and 400 ppm by volume or less.

27. A composition comprising: a trialkylamine in an amount of 99.9% by weight or more; and dimethylamine in an amount of 10 ppm by volume or less.

28. The composition according to claim 26 of 27, wherein all three alkyl groups in the trialkylamine are the same groups.

29. The composition according to claim 26, wherein the trialkylamine is trimethylamine.

30. The composition according to claim 27, wherein all three alkyl groups in the trialkylamine are the same groups.

31. The composition according to claim 27, wherein the trialkylamine is trimethylamine.

32. The method for purifying a trialkylamine according to claim 13, further comprising distilling a trialkylamine obtained by the contacting.

33. The method for purifying a trialkylamine according to claim 32, wherein the concentration of the dimethylamine in the crude trialkylamine is lowered to 400 ppm by volume or less in the contacting, and the concentration of the dimethylamine in the trialkylamine obtained by the contacting is lowered to 10 ppm by volume or less in the distilling.

34. The method for purifying a trialkylamine according to claim 33, wherein the trialkylamine is trimethylamine.

35. The method for purifying a trialkylamine according to claim 14, wherein the crude trialkylamine before contacting with the zeolite has a concentration of the dimethylamine of 500 to 1500 ppm by volume.

36. The method for purifying a trialkylamine according to claim 35, wherein the trialkylamine is trimethylamine.

37. The method for purifying a trialkylamine according to claim 34, wherein the crude trialkylamine before contacting with the zeolite has a concentration of the dimethylamine of 500 to 1500 ppm by volume.
